(19) United States
(12) Patent Application Publication
(10) Pub. No.: US 2007/0208389 A1
(43) Pub. Date: Sep. 6, 2007

(54) Coronary Sinus Locater Method and Apparatus for Biventricular Pacing

(76) Inventors: David C. Amundson, Boulder, CO (US); H. John Hanlin, Louisville, CO (US); Larry Blankenship, Boulder, CO (US)

Correspondence Address: PETER F WEINBERG;GIBSON DUNN AND CRUTCHER LLP, SUITE 4100, 1801 CALIFORNIA STREET, DENVER, CO, 80202, US

(21) Appl. No.: 10/593,438

(22) PCT Filed: Feb. 25, 2005
(86) PCT No.: PCT/US05/05813

§ 371 Date: Sep. 18, 2006

Related U.S. Application Data
(60) Provisional application No. 60/547,748, filed on Feb. 25, 2004.

(51) Int. Cl. A61N 1/00

(52) U.S. Cl. 607/27

(57) ABSTRACT

A coronary sinus locater sheath is stabilized in the vicinity of the coronary sinus by a guidewire exiting the side of the sheath near the distal end and inserted into an identifiable anatomical feature. The procedure includes (1) inserting the guide sheath into a vein, (2) advancing the sheath until the right atrium is entered, (3) advancing the sheath into the IVC, (4) extending a guidewire through a port near the sheath's distal end, (5) retracting the sheath above the IVC into the right atrium, (6) articulating or rotating the sheath until the sheath is near the coronary sinus ostium, while the sheath is stabilized by the guidewire. Thus the sheath can be positioned near the coronary sinus ostium limiting the motion imparted to the sheath from the beating heart. An imaging catheter may be used in conjunction with the sheath. The sheath or catheter may include pacing electrodes.

### RELATED US APPLICATION DATA

Applicant claims the benefit of Provisional patent No. 60/547,748 filed on Feb. 25, 2004.

### BACKGROUND OF THE INVENTION

I. Field of Invention

This invention relates to cardiac catheters/introducers used to access the coronary sinus and other anatomical features in the heart.

II. Related Art

Cardiac catheterizations are procedures in which a cardiologist inserts a catheter in the venous or arterial systems and navigates to the site of interest such as an artery, vein or chambers of the heart. In recent years, there has been increased interest in navigating the coronary sinus vasculature, particularly for the placement of permanent cardiac pacing and defibrillation leads such as those intended to pace the left ventricle. A new modality called biventricular pacing, has been developed which paces both the left and right ventricles to synchronize right and left ventricular contractions. One-third of patients with chronic heart failure have electrocardiographic evidence of a major intraventricular conduction delay, which may worsen left ventricular systolic dysfunction through asynchronous ventricular contraction. Studies suggest that biventricular pacing improves hemodynamics and well-being by reducing ventricular asynchrony. Biventricular pacing is accomplished by using a lead inserted in the coronary sinus to pace the left ventricle.

The coronary sinus vasculature wraps around the heart, with many branches lying laterally on the left ventricle in close proximity to ventricular muscle fibers. It is thus possible to pace these fibers in the left ventricle through an electrically conductive lead inserted from the right side of the heart. Permanent endocardial leads are only placed in the right heart, since implanted objects can produce an inflammatory response from the body, frequently with some thrombi formation. On the right side of the heart, a thrombi that has broken loose will travel to the lungs, with no deleterious effects. A thrombi formed in the left heart can travel to the brain, possibly producing a stroke. Consequently, pacemaker and defibrillator leads are implanted on the right side exclusively.

Currently, the coronary sinus is cannulated by introducing a deflectable or fixed-curve sheath or catheter into the right atrium and manipulating the sheath/catheter while viewing a fluoroscopic image until the cardiologist perceives entry into an orifice. Puffing dye and viewing the dye puff flowing into and then back out of the coronary sinus system on fluoroscopy make positive verification of entry into the coronary sinus. If a catheter is used, a sheath is then placed over the catheter and the catheter withdrawn. The sheath can then be advanced into a branch vein leading to the left ventricle. At this point, a guidewire can be inserted into the sheath to aid in navigation of a suitable venous branch adjacent to the left ventricle. The guidewire tip is floppy and can be manipulated to enter various branches of the coronary sinus vasculature. Once the proper branch is thought to be located, as evidenced by fluoroscopy, a pacing lead with a center lumen suitable for guidewire insertion is advanced over the guidewire to the desired location. At that point, pacing thresholds are determined, unwanted stimulation, such as of the phrenic nerve, is confirmed. If these conditions are acceptable the lead is connected to the implanted biventricular pacemaker. If they are not acceptable, the lead or guidewire is again manipulated to find a new location. This becomes an iterative procedure until acceptable pacing thresholds are obtained.

Within the first few centimeters into the coronary sinus, about a 90-degree turn is made to enter the coronary sinus branch, which traverses the left ventricle. About 3-6 cm beyond this turn, the posterior vein of the left ventricle branches off in about a 90-degree bend, near the anterior free wall of the left ventricle. It subsequently branches into lateral branches running down to near the anterior apex. Another 4-8 cm beyond the posterior vein branch the coronary sinus becomes the great cardiac vein, which branches in sharp bends to the antero-lateral branches. Both the posterior and antero-lateral branches are candidates for left ventricular pacing.

Pacing the left ventricle is accomplished by inserting a lead through the opening (ostium or os) of the coronary sinus and into a distal branch near left ventricular muscle fibers. Difficulties include:
1. Finding the opening (ostium) of the coronary sinus. Patients in CHF have hypertrophied hearts, which alters the location and size of the coronary sinus. Physicians routinely place leads in the coronary sinus during EP studies, however, they are dealing with normal-sized hearts with electrical conduction defects. With CHF patient candidates, finding the opening can be much more elusive. Sometimes it is located significantly off-center from the normal location since the heart has hypertrophied. Other times, flaps of tissue prevent entry into the coronary sinus.
2. Advancing the lead through the coronary sinus to a branch in close proximity to the left ventricle so it can be chronically paced. Pacemaker implants are performed on the right side of the heart since implants in the left heart could lead to thrombi heaving deleterious consequences such as a stroke or heart attack. The coronary sinus is the only area of the heart anatomy by which a lead can be inserted from the right heart into close proximity to the left ventricle. In fact, the tip of the pacing lead needs to be within several millimeters of ventricular muscle to successfully pace the ventricle. The coronary sinus branches into segments, five of which traverse the left ventricle. Locating the proper left-ventricular branch (where the left ventricle can be chronically paced) has been difficult in biventricular pacing clinical studies. Hypertrophied hearts also alter the location and length of these branches. Finding the correct branch in these highly variable hearts has been the other major challenge in biventricular pacing.

There have many disclosures of guide catheters and coronary sinus lead catheters that use pre-formed, deflecting and steerable curves to assist implantation into the coronary sinus os under fluoroscopy. Some disclosures attempt to provide guiding catheters (sheaths) or coronary sinus lead catheters with preferential curves of different flexibilities. This enables the physician to position the catheter near the coronary sinus where it is manipulated to enter the coronary sinus os. Such multi-radius curvature coronary sinus leads include Adams (U.S. Pat. No. 5,433,729), Lurie (U.S. Pat. No. 5,423,772), Tockman (U.S. Pat. No. 6,129,750) and Jaraczewski (U.S. Pat. No. 5,445,148). A variation to these curvatures is Swoyer (U.S. Pat. No. 5,683,445) who teaches a configuration with multiple 45 degree bends to position the electrode closely to the venous wall. Guiding catheters with angled curvatures include Randolph (U.S. Pat. No. 5,775,327), Lurie (USP App U.S.2002/0029030), and Toner (U.S. Pat. No. 5,488,960). A deflectable guide catheter is proposed by Williams (U.S. Pat. No. 6,408,214B1) in which a greater curvature can be achieved by pulling on a handle at the proximal end. A steerable, coronary sinus catheter is proposed by Ockuly (U.S. Pat. No. 6,458,107B1) in which the catheter is curved at steerable angles in one plane.

The purpose of the above inventions is to direct the catheter into the coronary sinus os, not to direct it into the appropriate branch of coronary sinus vasculature. Once in the coronary sinus vasculature, the guiding catheter would need to make approximately two 90 degree bends to reach a site appropriate for left ventricular pacing. Due to variations in the length of the vessels and the degree of bend in the branch points among hypertrophic heart patients, a fixed curved catheter would have the curve points and angles in the wrong place for the majority of patients. Even the deflectable catheter of Williams (U.S. Pat. No. 6,408,214B1) and the steerable, coronary sinus catheter as proposed by Ockuly (U.S. Pat. No. 6,458,107B1) are intended only to find the coronary sinus os by “touch and feel”, not to navigate in the coronary sinus vasculature through potentially tight, 90 degree bends. The curves described in these patents have a much too high radius of curvature to navigate within the coronary sinus branches usually used for long-term pacing.

Despite the existence of shaped guide catheters and leads, physicians commonly prefer to use a standard steerable EP ablation catheter, such as described by Avitall (U.S. Pat. No. 5,642,736). These catheters are favored to find the coronary sinus os, even though designed for mapping and ablation purposes, since physicians are familiar with the catheter's characteristics. In this approach, the physician inserts the steerable EP ablation catheter into the right atrium and then applies different curves to the distal end by manipulating controls on the proximal end. The catheter is usually dragged along the atrial wall until it encounters the coronary sinus os. Once the coronary sinus os is entered, a sheath is slid over the EP ablation catheter to cannulate the coronary sinus. The EP ablation catheter is then removed, leaving behind the sheath. The next steps depend on the configuration of the coronary sinus pacing lead. Some pacing leads have no guidewire channel, so once the sheath is in place, the coronary sinus lead is inserted through the sheath and manipulated using an internal stylet to enter the appropriate branch of the coronary sinus. Often, radio opaque dye is infused into the sinus and a snapshot is taken on the fluoroscopy machine to elucidate the branching points within the coronary sinus. Using the coronary sinus lead to access the proper branch can be difficult due to the size of the lead and the inability to make sharp-angled bends required to access a suitable coronary sinus branch.

Cardiac pacemaker manufacturers also offer coronary sinus leads with an open channel through the lead, through which a guidewire could be inserted. This permits the physician to find the coronary sinus branch with a small flexible guidewire, followed by insertion of the lead over the guidewire. When this system is used, following cannulation by the sheath, the guidewire is then inserted into the sheath and radio opaque dye is infused into the coronary sinus allowing a momentary picture of the coronary sinus vascular tree to be captured by the fluoroscopy camera. The physician then manipulates the guidewire to enter a branch suitable for long-term ventricular pacing. Once a site has been located, the coronary sinus lead is inserted over the guidewire and advanced until it occupies a suitable pacing site. Pacing and sensing thresholds are then taken to verify the coronary sinus lead is positioned to provide long-term left ventricular pacing for the patient. Once in proper position, the guidewire is removed and the lead proximal connector end is connected to the pacemaker.

The complexity in the curve geometries and stiffness characteristics of the above disclosures is due to the physician relying on “touch and feel” at the proximal end of the catheter. The various geometries place the coronary sinus guide catheter or lead in close proximity to the coronary sinus where small manipulations are only required to enter into the coronary sinus os. The difficulty with pre-curved catheters is the extreme variability of coronary sinus location and geometry in hypertrophic hearts. The entire heart and its internal structures tend to be distended by the growth of the heart. In addition, about 20% of the patients have tissue flaps over the coronary sinus, which prevent entry from certain directions. As a consequence of these limitations, implantation of a coronary sinus lead significantly increases the time of pacemaker implantation. A conventional right-sided pacer requires around an hour for implantation with over a 99% success rate. Biventricular pacers can require 3-6 hours implantation time, simply because of the difficulty in implanting the coronary sinus lead. Furthermore, the implantation success rate is only 80-90%, with cases abandoned because of inability to implant the coronary sinus lead. Moreover, following the implant, coronary sinus leads are much more prone to dislodgement. Reports suggest dislodgement rates of about 10-20% have been observed. Coronary sinus leads dislodge because anchoring means such as tines or screws, commonly used in the right atrium and ventricle, cannot be used in the coronary sinus. Stability is achieved by wedging the lead into a small branch to create a tight fit between the catheter and the coronary sinus branch. Dislodgement potential can be exacerbated by sub-optimal positioning in the coronary sinus system. The difficulty, frustration and time encountered in positioning the pacing lead in the iterative manner described above can lead to leaving the lead in the first location where acceptable pacing thresholds are achieved, even though it may not be the optimal position.

Recently, several real-time forward-imaging technologies have been developed which permit the physician to image the relation of the catheter to the os and branch points in front of the catheter. A forward-viewing technology can be a transducer near the distal end of the catheter, providing a view ahead of the catheter tip. For the purposes of this patent, forward-imaging is defined as imaging at an angle relative to the center axis of the catheter of less than 90 degrees which includes direct as well as off-angle forward imaging. Examples include near-infrared light Amundson (U.S. Pat. No. 6,178,346) and forward-imaging ultrasound such as Lin (U.S. Pat. No. 6,200,269). A forward-imaging technology is also providing local image enhancement at the catheter tip so that whole body real-time imaging can elucidate the relation of the catheter tip to the coronary sinus os or branch. An example is a modification of coronary sinus venography in which a radio opaque dye is infused in the coronary sinus and the heart region viewed with fluoroscopy. If the dye flows out through a lumen in the catheter tip for a long enough duration it becomes forward-viewing since it can be determined from whole body fluoroscopy where the catheter tip is located by observing the flow start point, and the vasculature ahead of the catheter tip. It becomes real-time since articulations of the catheter tip can be observed in the fluoroscopy monitor. Since the coronary sinus expels blood, the dye remains in the coronary sinus vasculature for only a brief instant and captured by the fluoroscopy camera. Recent developments include using a balloon expanded inside the os entrance to prolong the time for the dye to diffuse back into the right atrium.

Forward-imaging technologies in the form of a transducer in the catheter tip include disclosures by Amundson (U.S. Pat. No. 6,178,346) using near-infrared light, forward-viewing ultrasound such as Lin (U.S. Pat. No. 6,200,269), optical coherence tomography such as Wang (U.S. Pat. No. 6,041,248) and optical coherence domain reflectometry as described by Zeylikovich (U.S. Pat. No. 6,437,867). When a forward-imaging technology is included in the coronary sinus/branch-seeking catheter, different design considerations apply since the physician manipulates the catheter while observing an image on a monitor, which displays the structures in front of the catheter. This is most clearly demonstrated with near-infrared imaging (U.S. Pat. No. 6,178,346) in which a direct image is obtained, through blood, of the structures in the lower right atrium. This system uses near-infrared light 1600+/−70 nm to permit viewing through blood. Light is reflected off of the structure viewed, returning to the catheter where the reflected light is collected and transmitted to an infrared camera.

When near-infrared imaging is employed, the inferior vena cava appears as a large hole, the coronary sinus as a smaller hole. The tricuspid valve appears as a large hole with valve leaflets. Using these and other markers, a physician can direct the catheter so it is centered over the coronary sinus, and then push it through the coronary sinus os. Once in the coronary sinus, branches would appear as bifurcations and the branch entry hole or holes would be visible. Using other forward-viewing technologies, with image enhancement of holes present, might provide a similar picture.

Another real-time, catheter tip positioning technology that is somewhat different in nature is the lead navigation system, such as the CARTO system manufactured by Biosense Webster. These systems show the relationship of the catheter tip to the cardiac structure of interest. The Biosense/Webster system provides the six coordinates (x, y, z, yaw, pitch and roll) of a catheter containing a magnetic element and mapping electrode rings. By dragging this catheter on the cardiac interior, while simultaneously recording the electrical potentials at each point, a map of the cardiac interior can be obtained. Objects such as holes are recognized from the absence of electrical potentials and can be displayed as pictorial representations. The image, in this case, is a computer reconstruction illustrating the catheter position relative to perceived anatomical features, such as the coronary sinus.

In addition to this system, Medtronic manufacturers a lead locater system based on impedance and Boston Scientific has one based on ultrasound. All such systems require a locatable element in the catheter. In contrast to coronary sinus venography which can only image in the coronary vascular tree, systems like CARTO would only be useful in finding the coronary sinus os. These systems would not be useful in the coronary sinus vasculature, since the mapping catheter must first be in the vicinity of a structure to allow the system to map structure. These systems are useful only as feedback for finding the coronary sinus. However, in that respect they are no different than other feedback systems, they provide a real-time position of the catheter tip relative to anatomical features such as the coronary sinus os. Manipulations can be observed in the computer reconstructed illustration. These systems have not typically been employed to place coronary sinus catheters because of the length of time it takes to map the right atrium. More automated mapping may make these technologies candidates for feedback in coronary sinus catheter placement in the future.

Visual feedback alters the design considerations for guide catheters. The disclosures Adams (U.S. Pat. No. 5,433,729), Lurie (U.S. Pat. No. 5,423,772), Tockman (U.S. Pat. No. 6,129,750) and Jaraczewski (U.S. Pat. No. 5,445,148) all teach catheters which are designed with advantageous angled segments and flexibilities so that manipulation under fluoroscopy will successfully cannulate the coronary sinus os. In contrast, a guide catheter for an imaging device requires mechanical stability to minimize image movement. When the coronary sinus os and branches are imaged, the heartbeat tends to shake the catheter often leading to dislocation from the feature of interest as well as image smearing. One goal of a guide sheath, therefore, is to maintain catheter stability, which minimizes “jumping” of images and permits feature recognition.

### SUMMARY OF THE INVENTION

Locating the Coronary Sinus

Apparatus and means are disclosed for cannulating the coronary sinus with a sheath or catheter. The sheath or catheter has a port near the distal end where a guidewire can be advanced into an easily identifiable and accessible anatomical feature such as the inferior vena cava (IVC). The procedure is to advance the sheath/catheter (without guidewire advancement out of the port) into the inferior vena cava. Fluoroscopy, infrared endosocpic imaging, intracardiac echo or other means verifies placement in the inferior vena cava. Once in the inferior vena cava, the guidewire is advanced out of the port and the sheath/catheter is retracted into the right atrium, anchored and stabilized by the guidewire still residing in the inferior vena cava. Anchoring in the inferior vena cava stabilizes the sheath in a position near the coronary sinus. Small manipulations or rotation will orient the catheter/sheath near the coronary sinus. Once the coronary sinus is engaged, the guidewire is retracted from the inferior vena cava, allowing sheath/catheter to be further advanced through the coronary sinus vasculature.

With an anchored sheath, a variety of locater catheter/guidewire technologies can be used to locate the coronary sinus. They include:

1. An electrophysiology catheter

2. A guide sheath

3. A guidewire

4. Infrared imaging catheter

5. An electromagnetic mapping catheter (e.g. a magnetic catheter operating with the CARTO mapping system by Biosense Webster)

6. An intracardiac echo catheter

After the guide sheath containing one of the above devices is routed to the desirable location adjacent to the left ventricle, the following can

Once the sheath is guided to the proper be utilized:

Pacing Threshold Evaluation location in the coronary sinus vasculature, pacing thresholds as well as deleterious pacing effects such as phrenic nerve stimulation can be assessed by having two rings on the sheath or catheter placed through the sheath (e.g., an imaging catheter) mimicking pacing electrodes. These electrodes are connected by wires to outside the patient. They are connected to a pacing threshold analyzer. The analyzer is used to determine pacing threshold and phrenic nerve and muscle stimulation potential.

Implantation of Pacing Lead

After a site is examined for its suitability as a pacing site, the catheter is removed. A guidewire is inserted through the sheath and a coronary sinus pacing lead is inserted over the guidewire to the distal end to the same location where successful pacing was observed. The sheath and guidewire are then removed leaving the pacing lead in a position suitable for long term pacing. After pacing threshold evaluation, the lead is then connected to the implanted biventricular pacemaker.

### BRIEF DESCRIPTION OF THE DRAWINGS

FIG. 1 is a drawing of the guide sheath placed in the right atrium

FIG. 2 is a drawing of a guide sheath containing a catheter encountering a branch point in the coronary sinus vasculature

### DETAILED DESCRIPTIONS OF THE EMBODIMENTS

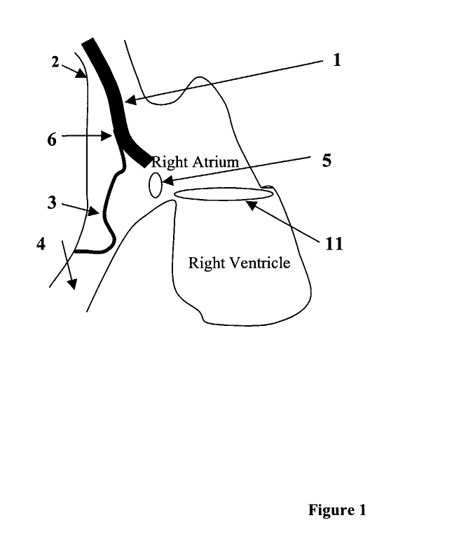

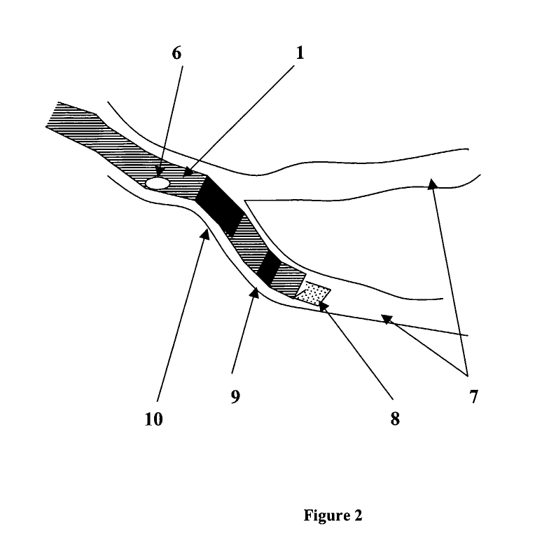

This embodiment preferably uses the infrared imaging catheter described in U.S. Pat. No. 6,178,346. The infrared imaging catheter is inserted in a sheath (1) shown positioned in FIG. 1 containing the guidewire port (6) (also see FIG. 2) about 3-10 cm from the distal end of the sheath. The sheath-catheter (1) also has two electrodes (9, 10) near the distal end as shown in FIG. 2. The most distal electrode (9) is about 10 square millimeters in surface area. One centimeter back from the most distal electrode is the proximal electrode (10) about 30 square millimeters in surface area. Both electrodes are connected to insulated wires, which extend out the proximal end of the sheath for connection to a pacing analyzer.

Using fluoroscopy or the infrared images, the catheter-sheath is guided into the inferior vena cava. Once in the inferior vena cava, the guidewire (3) (see FIG. 1) is then extended. The guidewire (3) may be a traditional guidewire used in navigating vasculature structures or may be optimized to anchor and stabilize to the structure. Once the guidewire (3) is extended, the sheath-catheter is retracted out of the inferior vena cava by sliding proximally along the guidewire, which still in place inside the inferior vena cava. The guidewire (3) now serves as an anchor for the sheath-catheter (1). FIG. 1 shows the configuration with the guidewire (3) extended into the IVC (4) and the catheter-sheath positioned directly above the coronary sinus (5).

The sheath-catheter (1) is then articulated and rotated until the CS comes into view of the imaging catheter. Specific techniques can be developed, with or without the guidewire, to manipulate the sheath-catheter, visually locating easily identifiable anatomical features, such as the tricuspid valve (11) (see FIG. 1), and using such features and landmarks to locate the coronary sinus ostium. Once the coronary sinus ostium is in view, the sheath-catheter is advanced into the coronary sinus. Alternatively, the sheath-catheter could have a fixed curve and be rotated to find the coronary sinus. This could be accomplished several ways. The catheter (8, FIG. 2) may be telescoped from the sheath into the coronary sinus. The catheter may also be permanently attached to the sheath. Once the coronary sinus has been engaged, the guidewire is withdrawn out of the inferior vena cava and the imaging catheter can be used to navigate the coronary sinus vasculature. As the sheath-catheter is advanced, branches will be encountered. The sheath-catheter (1) can be moved (such as being rotated or articulated) to enter a particular branch.

Once the sheath-catheter has reached a desirable pacing site, the sheath-catheter electrode wires are connected to a pacing analyzer and pacing thresholds are obtained. Phrenic nerve stimulation is evaluated by pacing at 10 volts. If suitable thresholds are found with no phrenic nerve stimulation, the imaging catheter may be withdrawn. A guidewire may then be inserted through the sheath and routed beyond the distal end of the sheath. Over the guidewire, a coronary sinus pacing lead may be inserted and advanced to the end of the sheath so that the pacing electrodes are adjacent to the electrodes on the sheath. The sheath is withdrawn and pacing thresholds are again obtained with the pacing lead. If still acceptable, the lead is connected to a biventricular pacemaker.

In summary, this invention teaches a method of placing a sheath in a stable position near a structure in the heart using an anchoring technique of inserting a guidewire into a nearby anatomical features, such as the IVC. The embodiments teach the cannulation of the coronary sinus with a sheath-catheter by anchoring in the inferior vena cava with an extendable member from a port in the sheath. This places the sheath-catheter in the vicinity of the coronary sinus. The coronary sinus is then engaged and the extendable member is then retracted from the IVC to permit further advancement into the coronary sinus vasculature. Once an appropriate pacing site is found, the pacing viability can be assessed by pacing with electrodes located near the distal end of the sheath-catheter. If acceptable thresholds are obtained, the catheter is removed and replaced with a pacing lead. Finally, the sheath is retracted leaving the pacing lead in place ready for connection to the biventricular pacemaker.

1. A method of placing a sheath in a stable position in or near a desired anatomical structure in a heart chamber having a known nearby anatomical feature, comprising the steps of:
inserting the sheath into the heart chamber;
positioning the sheath with respect to the nearby anatomical feature;
extending a guidewire from the sheath into or near to the nearby anatomical feature;
retracting the sheath while the guidewire is extended into or near to the nearby anatomical feature; and
moving the sheath to place it in or near the desired anatomical structure.

2. The method of claim 1, wherein the sheath is deflectable.

3. The method of claim 1, wherein the sheath is a fixed-curve and torqueable.

4. The method of claim 1, wherein the desired anatomical structure is the coronary sinus.

5. The method of claim 1, wherein the nearby anatomical feature is the inferior vena cava.

6. The method of claim 1, further comprising imaging the anatomical structure with an imaging catheter contained at least partially within the sheath.

7. The method of claim 1, further comprising the steps of disengaging the guidewire and then further moving the sheath.

8. The method of claim 1, further comprising the step of determining pacing thresholds using pacing electrodes positioned on or near the sheath.

9. The method of claim 8, further comprising the step of introducing a pacing lead into the heart chamber.

10. A method of placing a sheath within a patient's vasculature, comprising:
advancing the sheath within the vasculature;
recognizing a first anatomical feature;
stabilizing the sheath by attaching it to or near the first anatomical feature; and
positioning the sheath to locate a second anatomical feature.

11. The method of claim 10, further comprising the steps of disattaching the sheath from first anatomical feature, and further positioning the sheath.

12. The method of claim 10, wherein the sheath is positioned at least in part in connection with imaging means on or within sheath.

13. The method of claim 10, further comprising the step of determining pacing thresholds.

14. Medical apparatus for placement in a stable position in or near a desired anatomical structure with a patient's vasculature comprising:
a sheath for inserting into a patient's vasculature, the sheath having a port near a distal end of the sheath;
a guidewire at least partially contained within the sheath, the guidewire being capable of being extended through the port and attaching to the vasculature; and
whereby the sheath can be retracted from guidewire and further positioned within the vasculature.

15. The apparatus of claim 14 wherein the sheath is deflectable.

16. The apparatus of claim 14 wherein the sheath is fixed-curve and torqueable.

17. The apparatus of claim 14 wherein the sheath is capable is capable of being introduced into the coronary sinus.

18. The apparatus of claim 14 wherein the sheath includes electrodes at or near the distal end for sensing pacing viability at potential pacing site.

19. The apparatus of claim 14 wherein the sheath includes an imaging catheter.

20. The apparatus of claim 14, further comprising means for introducing a pacing lead into the patient.
